(19) United States
(12) Patent Application Publication
(10) Pub. No.: US 2006/0216811 A1
(43) Pub. Date: Sep. 28, 2006

(54) Use of bacteria to prevent gas leakage

(76) Inventors: Alfred B. Cunningham, Bozeman, MT (US); Lee H. Spangler, Bozeman, MT (US); Robin Gerlach, Bozeman, MT (US); Adrienne J. Phillips, Bozeman, MT (US)

Correspondence Address: COOLEY GODWARD LLP, THE BROWN BUILDING - 875 15TH STREET, NW, SUITE 800, WASHINGTON, DC, 20005-2221, US

(21) Appl. No.: 11/346,285

(22) Filed: Feb. 3, 2006

Related U.S. Application Data
(60) Provisional application No. 60/649,802, filed on Feb. 3, 2005.

(51) Int. Cl. C02F 3/34; C12P 1/04

(52) U.S. Cl. 435/262; 435/41; 435/170

(57) ABSTRACT

The present invention relates to the use of microbial biofilms and microbial induction of calcium carbonate precipitation to sequester gases in underground geological formations. In one embodiment, methods of the invention can be used to prevent the leakage of supercritical CO₂ in underground geological formations such as aquifers.

### CROSS-REFERENCE TO RELATED APPLICATION

This application claims benefit of U.S. Provisional Application No. 60/649,802, filed on Feb. 3, 2005, which is herein expressly incorporated in its entirety.

### FIELD OF THE INVENTION

The present invention relates to methods of sequestering gases such as CO₂ in geological formations with minimal gas leakage by applying microbial biofilms.

### BACKGROUND OF THE INVENTION

There is mounting and compelling evidence that anthropogenic green house gases (GHG) are generating climate change. The Intergovernmental Panel on Climate Change (IPCC) credits anthropogenic GHGs with causing a 0.6±0.2° C. increase in the global mean surface temperature over the 20th century, a 5 to 10% increase in continental precipitation, and a decrease in frost days. Additionally, GHGs are likely responsible for an increase in heavy precipitation events in some regions and frequency and severity of droughts in others.

The rise in mean global temperature tracks the anthropogenic emission of green house gases with CO₂ being the major constituent. CO₂ is blamed for approximately 64% of the anthropogenic greenhouse effect. Ice core samples and direct atmospheric measurements indicate that the CO₂ concentration remained roughly constant at 280 parts per million volumes (ppm) from the years 1000 to 1780 but had risen to 368 ppm by the year 2000. While other GHGs have shown similar (e.g. N₂O) or larger (e.g. CH₄) percentage increases, their atmospheric concentrations are 200 to 1000 times lower than that of CO₂. Concerns over the emission of GHG such as CO₂ have resulted in President Bush's Global Climate Change Initiative which calls for an 18% decrease in GHG emissions by 2012.

CO₂ emissions are most directly tied to use of fossil fuels as an energy source with the oxidation of carbon (along with hydrogen in hydrocarbon fuels) being the fundamental chemical process responsible for the release of energy. With the global natural gas reserves being expected to dwindle on the 10 to 20 year timeframe and the oil reserves projected to deplete on the 25 to 50 year scale, coupled with the desirable goal of increased energy independence, carbon intensive coal is likely to play an increasing role in the US energy portfolio. Global and US coal reserves have a projected 210 year duration buying valuable time for development of other energy resources, but potentially exacerbating the GHG and climate change issues unless technologies and procedures are developed for CO₂ mitigation.

In order to have a large initial impact, it is sensible to focus on point sources of CO₂ emissions. While all major point sources should be considered, power generation from fossil fuels is particularly noteworthy given that it is responsible for approximately 7.7 Gtons CO₂ per year, roughly 37% of CO₂ emissions. Because CO₂ is a natural product of energy generation from fossil fuels, there is no way to prevent its production (although efficiency improvements do reduce emissions), and mitigation of CO₂ in the fossil power production process must come either through chemical conversion of the CO₂ or through sequestration. Fundamentally, reduction of the CO₂ to form useful hydrocarbons (such as polymers) consumes energy amounts similar to the amount of energy liberated by oxidation during fuel utilization making this option impractical on the scales needed to have a major reduction of emissions. Accordingly, sequestration as the only viable option to reduce CO₂ emissions.

The three major options for storage and sequestration are oceanic sequestration, terrestrial sequestration and geologic sequestration. Oceanic sequestration involves pumping CO₂ into the ocean as a droplet plume in (a) an unconfined release below 1000 m; (b) a confined release at depths greater than 1000 m where the temperature and pressure are appropriate for formation of a CO₂ and H₂O clathrate (a solid hydride); or (c) very deep release below 3000 m thereby forming a “CO₂ lake”. The potential for ocean storage is tremendous making this an important area for research. However, the long term ecological impacts of ocean sequestration are not well known, as a result, this is not a good option for large scale CO₂ mitigation in the near term.

Terrestrial storage involves the uptake of atmospheric carbon in soil and in plant biomass. While this is an attractive near term approach that can capture carbon directly from the atmosphere, it has limited total capacity and permanence of storage is a major issue.

Geologic storage involves pumping CO₂ underground into formations that will result in sequestration. Geologic storage is an exciting avenue for CO₂ mitigation because it places the carbon back into the part of the geosystem from where it originated (albeit in a different chemical form) which may reduce geosystem impact. This option has additional attractive features such as the potential to co-locate the sequestration with the source reducing CO₂ transport issues and cost as well as the potential to store CO₂ with very low leakage. Further, geologic storage may be able to provide a very large storage capacity.

### SUMMARY OF THE INVENTION

The present invention includes a method of using a microbial biofilm barrier to seal a gas leakage in a geological formation containing sequestered gas comprising applying one or more microbial species to the geological formation followed by the administration of a growth substrate. In one embodiment of the invention, the gas is supercritical CO₂.

The microbial species of the invention are capable of surviving and growing in underground geological formations, including, but not limited to, aquifers, deep coal beds, depleted oil reservoirs, depleted natural gas reservoirs, and salt caverns. Accordingly, it is preferred that the one or more microbial species be capable of growth at 40° to 60° C., about 5% salinity and about 100 atmospheres of pressure. In one embodiment of the invention, native microbes, i.e., microbes that are naturally found in underground geological formations, are applied in a geological formation for the induction of a biofilm barrier/calcium carbonate precipitation barrier. For instance, Shewanella sp. can be used with the methods of the present invention.

In one embodiment of the invention, the biofilm barrier of the methods of the invention reduces leakage of sequestered gas through aquitard traps associated with aquifers. The biofilm barrier of the invention can also be used to mitigate the leakage of gas that has already seeped out of an aquitard and has the potential for contaminating surrounding strata.

The methods of the invention can also be used to reduce leakage of sequestered gas from an aquifer. In one embodiment, the biofilm reduces leakage of sequestered gas through one or more aquitard traps. The biofilm of the invention is able to reduce leakage of sequestered gas by the reduction of permeability of the geological formation.

### BRIEF DESCRIPTION OF THE DRAWINGS

FIG. 1 is a diagram of a high pressure core testing system.

FIG. 2 is a Scanning Electron Microscope (SEM) image showing biofilm clusters on the mineral surface of brea sandstone core.

### DETAILED DESCRIPTION OF THE INVENTION

The inventors of the present invention have developed methods for sequestering gases such as CO₂ in geological formations by inducing the growth of biofilm. The biofilm functions as a seal to stop and prevent leakage of sequestered gas. In one embodiment of the invention, a microbial biofilm barrier functions to mitigate gas leakage in surrounding strata.

Attractive aspects of biofilm barrier technology for enhancing geologic sequestration of gases include: 1) biofilm barrier construction can be achieved without excavation and therefore may be useful at sites where access to the subsurface is restricted; 2) there is no obvious depth limitation with biofilm barrier technology; and 3) once established, the biofilm barrier requires minimal maintenance for long-term operation.

Microbes

As used herein, “microbial species” refers to all types of microbes capable of forming a biofilm. In one embodiment, the microbial species is a bacterial species. For instance, Shewanella sp. can be used with the methods of the present invention.

As can be appreciated by a skilled artisan, a biofilm of the invention can be created by adding exogenous microbes to a geological formation and inducing biofilm formation, by adding nutrients to endogenous microbes already present in a geological formation to induce biofilm formation, or by adding a biofilm to a geological formation.

The microbial species of the invention can be native to an underground geological formation. As used herein, “native” means that the microbial species are capable of being naturally isolated from a geological formation. For clarity, native microbial species do not have to originate from the same geological formation to which they are being added. The term native implies that the microbial species are naturally evolved to survive in an underground geological formation.

Microbial species of the invention are preferably capable of growth at about 40° to 60° C., about 5% salinity and about 100 atmospheres of pressure. Temperatures for applications of biofilm barriers for supercritical CO₂ injection range from approximately 40 to 60° C. This is within the growth temperature range of moderately thermophilic bacteria (extreme thermophiles have optimal growth temperatures above 80° C.). A wide variety of bacteria can grow at moderately thermophilic temperatures. Pressures expected in supercritical CO₂ injection are more moderate (about 100 atmospheres) and require moderately barophilic or barotolerant bacteria.

In one embodiment of the invention, microbes are able to seal a leak or prevent leakage of a sequestered gas by inducing calcium carbonate precipitation. The calcium carbonate precipitant can be part of the microbial biofilm. The use of biofilm as used herein is inclusive of a calcium carbonate precipitant component.

Biofilm

The methods of the present invention involve the application of one or more microbial species to a geological formation to form a biofilm barrier. As used herein, microbial species refers to all types of microbes capable of forming a biofilm. In the preferred embodiment, the microbial species is a bacterial species.

As used herein, “biofilm” is a matrix of microbial cells and extracellular polymers. A biofilm is a community of microorganisms, either single or multiple microbial species, that adhere to a substrate in an aqueous environment. “Biofilm” and “biofilm barrier” are used interchangeably herein. A biofilm can be formed by a single bacterial species, but more often biofilms consist of many species of bacteria, as well as fungi, algae, protozoa, debris and corrosion products. Biofilm bacteria execrete extracellular polymeric substances which act to anchor them to substrates, including, but not limited to, metals, plastics, soil particles, medical implant materials, and tissue. Essentially, biofilm may form on any surface exposed to bacteria and some amount of water. Once anchored to a surface, biofilm microorganisms carry out a variety of detrimental or beneficial reactions.

Biofilm barrier technology involves the injection and subsurface transport of starved bacterial cultures followed by resuscitation with injected growth substrates as known in the art. As used herein, “nutrient substrate” refers to growth enhancements and nutrient and supplement mixtures and the like.

Geological Sequestration

The basic principle behind geologic sequestration is the injection of supercritical CO₂ into underground geologic formations. Supercritical (temperature Tc=31.1 C, and pressure Pc=7.38 MPa) CO₂ is injected so that the density of CO₂ is high and more CO₂ can be stored per unit volume available.

Geological Formations

As used herein, “geological formations” refer to man-made and natural structures that can serve as underground reservoirs. Gases such as CO₂ can be sequestered through geological formations including, but not limited to, enhanced oil recovery (EOR), depleted oil and gas reservoirs, deep coal beds (ECBMR), deep saline aquifers and salt caverns.

Deep Saline Aquifers

An aquifer is a formation, group of formations or part of a formation that contains sufficient saturated, permeable material to yield significant quantities of water. Aquitards are associated with aquifers. Unlike an aquifer, an aquitard is not permeable enough to yield significant quantities of water.

Because deep saline aquifers exist in large regions throughout the United States and the world, this geology has attracted significant attention. Because of their ubiquity, one estimate states that up to 65% of CO₂ production from US power plants can be injected into deep saline aquifers without long pipeline transport. Estimates of worldwide capacity range from 350 to 11,000 Gt CO₂ and US capacity estimates range from 5.5 to 550 Gt.

There are several general conditions that must be met for sequestration in these formations: 1) the temperature and pressure conditions must be such that the CO₂ will be supercritical; 2) the aquifer must have a suitable aquitard trap; and 3) the aquifer should have appropriate porosity and permeability. Generally, depths greater than 800 m provide pressures above the supercritical pressure and, in many cases, will have a high enough temperature. It has been suggested, however, that geothermal gradients can vary significantly even within the same basin and required depths can be less than 700 m to >1200 m (Bachu).

Once injected, there are three main trapping mechanisms that can occur: solubility trapping; hydrodynamic (or structural) trapping; and mineralization trapping.

Solubility trapping occurs when the CO₂ is dissolved in the brine. However, solubility decreases as salinity increases typically resulting in two fluid phases. While this, in principle, could store large quantities, dissolution is not expected to be rapid and much of the CO₂ could remain in a separate phase for long times.

Hydrodynamic trapping was first proposed by Bachu. When the carbon dioxide is outside the injection well radius of influence, it will travel at the same velocity as the regional aquifer flow system. These flow rates for deep aquifers are on a geological time scale, 1-10 cm/yr meaning that the CO₂ will remain within tens of kilometers of the injection site over a million year time scale.

In both solubility and hydrodynamic trapping cases, porosity and permeability of the aquifer play a critical role. Porosity must be high enough to provide significant storage potential in the aquifer. Permeability should be high near the well site to permit injection of large quantities, but regional permeability should be low enough to ensure long residence times for hydrodynamic flow.

Mineralization trapping involves formation of carbonates via geochemical reactions that can result in very stable sequestration. The main steps in the process are dissolution of CO₂ in water to form bicarbonate and subsequent reaction of the carbonate with Ca2+, Fe2+, Mg2+, or (Mg2++Ca2+) to form the carbonates siderite, magnesite or dolomite, respectively. Mineralization trapping can lock the CO₂ into a very stable carbonate that would not leak.

Naturally occurring reservoirs of CO₂ exist and the McElmo Dome in Colorado and the Bravo Dome in New Mexico have been the subject of significant study. Detailed analysis of Bravo dome core samples indicates dissolution of dolomite and anhydrite and precipitation of kaolinite, zeolites, and gibbsite resulting in a decrease in formation permeability. Attempts to reproduce the geochemistry in the lab failed and was taken as an indication the rates of the processes were slow.

Aquifer storage has been demonstrated on an industrial scale since 1996 in the Sleipner natural gas field off the coast of Norway, where 1.1 Mt CO₂/yr is injected below the seabed into an aquifer 1000 m deep. The operation is economically feasible because of Norway's steep CO₂ emission tax. Other evaluation projects are underway to identify potential sequestration pilots including the Frio formation on the Gulf coast of Texas, and the Mt Simon formation near the Ohio-West Virginia border. The occurrence of stable natural analogs plus these projects, combined with the ubiquity of deep saline aquifers, speak to the promise of geologic storage, but there are potential problems and numerous issues that must be addressed.

Supercritical CO₂ injected into a receiving formation can result in elevated pressure in the region surrounding the point of injection. As a result, an upward hydrodynamic pressure gradient can develope across the trapping aquitard. Upward “leakage” of CO₂ can subsequently occur due to the primary permeability of the aquitard or through fractures. Additional concerns include induced seismicity, environmental aspects, and permanence of storage. In one embodiment of the invention, microbes are engineered to form biofilms for reducing the leakage of supercritical CO₂ through aquitards, as well as restricting the migration of CO₂ leaks which have penetrated through aquitards.

The methods of the present invention address the concerns discussed in detail below which are related to use of aquifers for storage of supercritical CO₂. Methods of the present invention use microbial species to produce copious amounts of extracellular polymer (EPS), which plug the free pore space of the aquifer thereby reducing porosity and hydraulic conductivity. This zone of reduced hydraulic conductivity forms a barrier for the supercritical CO₂.

Induced Seismicity

Mechanical failure of rock can be induced via injection due to either increased stress or reduction in strength caused by fault zone lubrication. An internet bibliography of injection induced seismic events has been compiled and the occurrence of multiple events indicates that this is an important issue in risk assessment of geologic sequestration. Over 20 articles in the bibliography are devoted to earthquakes induced by wastewater injection into a 3.7 km well associated with the Rocky Mountain Arsenal near Denver. Starting in 1962, Denver has experienced more than 700 earthquakes (in an area with no previous seismic activity) that initiated one month after injection began, with one event estimated at 5 on the Richter scale. A very clear correlation between injection and seismic events has been established and epicenters were determined to be within 11 km of the well. The probability that these events were naturally occurring and unrelated to injection was estimated at 1 in 2.5 million. This methods of the present invention may be used to stabilize rock formations and thus reduce the likelihood and/or occurrence of seismic activity related to injection of gases.

Environmental Aspects

While CO₂ is relatively non-toxic, it is heavier than air and, although highly unlikely, a catastrophic leak resulting in a large, fast release of CO₂ could result in asphyxiation. Migration of CO₂ to shallower drinking water aquifers can produce pH changes in the water and result in dissolution and mobilization of trace metals, metalloids and radionuclides. Methods of the present invention can be used to reduce the risk to people and animals of a catastrophic CO₂ leak.

Permanence of Storage

If the goal is to mitigate greenhouse gases, it is clear that if significant leakage occurs on a human timescale, geologic storage will not provide as useful a solution to climate change. Anderson and Vogh surveyed storage of methane in 229 reservoirs by 87 companies in depleted oil fields, aquifers, and salt caverns. Losses were reported by 37 companies with most being minor but four massive and uncontrollable losses were experienced. Given DOE goals for retention levels of stored CO₂, losses characterized as minor may be above suitable GHG leakage levels. Aquifers exhibited a “significantly higher incidence of serious gas loss than the other reservoir types”.

Principal modes of CO₂ leakage from brine formations include integrity failures of the injection well or abandoned wells in the vicinity; natural or stimulated seismic events, allowing buoyant CO₂ to escape upward via fractures, and caprock integrity failure through chemical degradation of the formation minerals or mechanical changes due to increased formation pressures. Leakage of geologically sequestered gases falls under three broad categories distinguished by the mechanisms, pathways, and quantities (fluxes and concentrations) of CO₂ involved: acute leakage, diffuse leakage and microseepage. Five important pathways for acute and diffuse leakage of CO₂ to the atmosphere are (1) vertical migration through fractures in the caprock; (2) buoyancy driven flow through permeable zones of caprock; (3) leakage of CO₂ through the weilbore (blowout); (4) escape through the well casing to thief zones in the overburden, and subsequent bubbling from these collector zones to the surface; and (5) diffusion as a dissolved phase through a water saturated caprock. The methods of the present invention can be used to target the above described pathways for acute and diffuse leakage of CO₂.

Other Geologies

Other promising storage geologies include injection into oilfields for enhanced oil recovery (EOR) and injection into deep, unminable coal seams possibly coupled with enhanced coal bed methane (ECBM) production. Worldwide, there are over 70 EOR projects and global capacity is estimated at 73-238 Gt CO2.50. To date, projects have focused on oil extraction due to economic benefit and percentage of CO₂ sequestered and permanence of storage should be investigated for these sites. Storage in coal beds is of interest as well. Carbon dioxide has a greater affinity for coal than does methane with approximately twice as many CO₂ molecules adsorbed in a given volume of coal compared to CH₄. This means that injection of CO₂ into coal beds can result in stable sequestration and potentially liberation of existing coal bed methane. Estimates of global storage capacity range from 300-900 Gt CO₂. However, there is a very incomplete understanding of the geochemical and geophysical processes caused by injection of CO₂ into coal.

The biofilms of the present invention can be used with these technologies to reduce the leakage of CO₂ and other gases. As with aquifers, one or more microorganisms can be applied to these geological formations and biofilms induced.

Subsurface Biofilm Barrier Concept

In one embodiment of the invention, biofilm containment barriers are formed by injecting selectable bacteria and transporting it through the subsurface between adjoining injection wells. After the formation has been inoculated with starved bacteria, suitable growth substrate and nutrients are injected to stimulate microbial growth and biofilm formation. The main advantages offered by biofilm barrier technology are: 1) biofilm barrier construction will be achieved without excavation and therefore may be potentially useful at locations with restricted access to the subsurface, and 2) there is no obvious depth limitation with biofilm barrier technology.

Bacteria for Supercritical CO2 Barrier

Biofilm barriers have been formed using a variety of bacterial species and conditions. Initial experiments were performed by the inventors of the present invention with an oilfield isolate of Klebsiella o.x-ytoca under fermentative conditions and mesophilic temperatures. Barriers have been formed both in the laboratory and in field tests with Pseudornonas fluorescens under denitrifying conditions at lower temperatures (ca. 10° C.). For microbial enhanced oil recovery (MEOR) the inventors of the present invention have previously formed barriers with Agrobacterium radiobacter under denitrifying conditions and mesophilic temperatures (20-25° C.).

Temperatures for applications of biofilm barriers for supercritical CO₂ injection range from approximately 40 to 60° C. This is within the growth temperature range of moderately thermophilic bacteria (extreme thermophiles have optimal growth temperatures above 80° C.). A wide variety of bacteria can grow at moderately thermophilic temperatures.

The use of microbial biomass to plug free pore space in porous matrices was first exploited for microbial enhanced oil recovery by Jack, et al. Research related to this application revealed that the production of extracellular polymers by bacteria was an important factor in permeability reduction and surveys were conducted to isolate polymer-producing bacteria that were tolerant of the high temperatures and salinities found in oil reservoirs. Enrichments conducted at moderately high temperature (50° C.) and salinity (5%) produced a variety of isolates that produced extracellular polysaccharides. In a more recent survey of bacteria in high temperature petroleum reservoirs, a variety of fermentative bacteria were isolated from production waters of petroleum reservoirs with depths ranging from 396-3048 m, temperatures ranging from 21-130° C., and salinities ranging from 2.8-128 g/L. Thermophilic fermentative microorganisms were successfully isolated from all production waters with salinity values of 40 g/L or less.

In addition being able to grow at high temperatures and salinities, many species of microorganisms are adapted to growth at high pressures. Extremely barophilic microorganisms can grow at pressures exceeding 1000 atmospheres. These bacteria were isolated from deep ocean trenches. Bacteria isolated from deep-sea hydrothermal vents are capable of growing under extreme conditions of both temperature and pressure. Pressures expected in supercritical CO₂ injection are more moderate (−100 atmospheres) and require moderately barophilic or barotolerant bacteria. During pilot scale testing of MEOR, we have injected a polymer-producing strain of Agrobacterium radiobacter into an oil reservoir at pressures of over 100 atmospheres. This strain was isolated from water produced by the reservoir but was not specially selected for barotolerance. Overall, this research indicates that the temperature, salinity, and pressure expected in supercritical CO₂ injection applications will allow the formation of biofilm barriers using appropriate species of bacteria.

### EXAMPLES

1. Development of Mesoscale Packed Column System for Evaluating Biofilm Plugging in Porous Media Representative of Field Conditions

Methods

Microbial inocula were screened to assess their potential to form biofilm barriers under relevant temperature and salinity conditions (data not shown). A laboratory method was then developed to screen microbial cultures for acceptability in forming thick, mucoid biofilms in porous media. A bench-scale core testing system was constructed to screen various microbial inocula for their biofilm formation properties in porous media along with their growth kinetics under various temperature, salinity, and pressure drop conditions. Microbial inocula can be screened as follows: a packed bed column is inoculated with starved bacteria. After inoculation, the packed bed column is fed an appropriate growth substrate and nutrients to promote biofilm accumulation in the column. As biofilm develops, the corresponding pressure drop and flow rate across the reactor can be continuously monitored. Changes in media hydraulic conductivity, permeability and porosity are subsequently computed. Temperature can be controlled by setting up the column system in controlled incubator or bench-top environments. A glass capillary tube containing model porous media can be run in parallel with the packed column. This capillary will facilitate the growth and accumulation of biofilm in a manner that can be imaged microscopically thereby facilitating visual analysis of the biofilm. The biofilm on the porous media can be recovered at the conclusion of each experiment and subjected to tests to determine EPS production and biomass formation.

Results

Using the above described mesoscale packed column system, an oil field isolate, Shewanella frigidamarina, was identified as a good biofilm former when grown under denitrifying conditions with a Brain-Heart Infusion media. The resulting biofilm lowered the permeability of 0.5 mm glass beads by more than two orders of magnitude (additional data not shown).

2. Development of a High Pressure Core Testing System

Methods

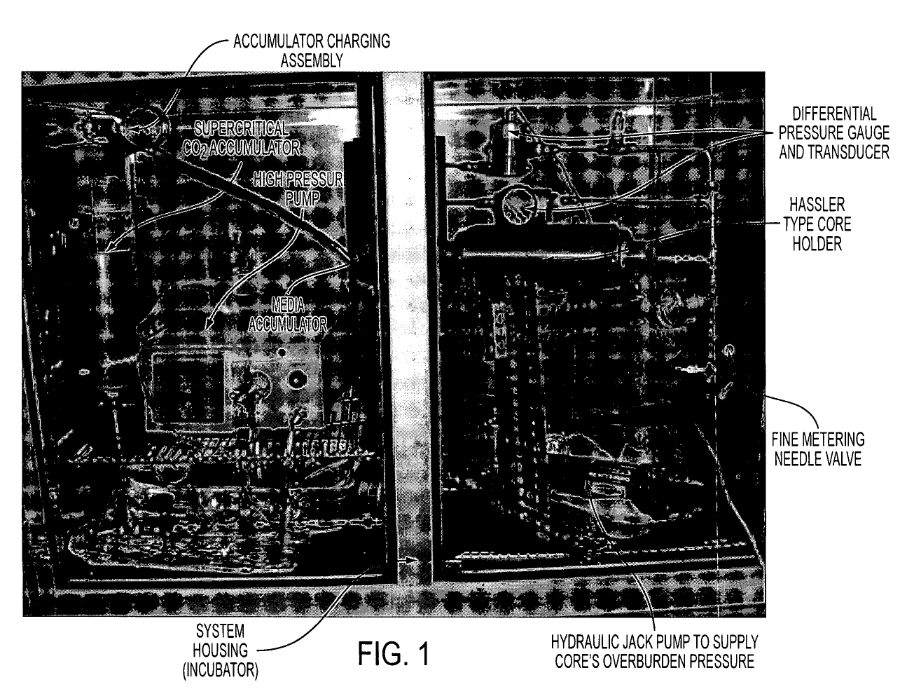

A high pressure core testing system was developed as is shown in FIG. 1. This system facilitates the growth of microbial biofilms in one-inch diameter sandstone cores under pressures in excess of 1200 psig and temperatures of 20° to 40° C. After biofilm has been developed, the cores can be challenged with supercritical CO₂.

This high pressure system is comprised of 2 Parker piston type accumulators (one for media and one for supercritical CO₂ storage), a high pressure pump (Accuflow Series III) to fill liquid CO₂ into the accumulator, a Hassler type core holder (Temco), a differential pressure gauge and a differential pressure transducer (Setra Model 235), an effluent fine metering control valve (Swagelok), and a balance (Sartorius T2101) to collect effluent mass data. Several check, purge, and shutoff valves (Swagelok) are designed in the system to allow for component isolation, bleed air out of the system, or to release system pressure quickly if needed. The high pressure core testing system was built inside of an incubator to reach supercritical conditions for CO₂ and as secondary containment to prevent high pressure accidents.

All of the system components were rated to at least 2000 psi and comprised of stainless steel. ¼″ stainless steel tubing was used to connect the components with the exception of the liquid CO₂ influent line to the high pressure pump which was comprised of ⅛″ PEEK tubing for ease of connection to the pump. FIG. 1 shows the system setup with the labeled components.

System pressure is controlled with the accumulator charging assembly that is attached to a high pressure regulator on a nitrogen tank outside of the incubator and the fine metering needle valve on the liquid effluent side of the system. After a setup period of purging off the air in the system, the fine metering needle valve is used to set an initial flow rate of fluid through the core.

The balance collects liquid effluent mass and time data with the use of an RS-232 output and a Labview VI. The differential pressure transducer outputs voltage measurements to a DATAQ data-logger that records the raw voltage data. The voltage data is then converted to pressure with the calibration curve provided by Setra.

The high pressure core pressure system works with the packed column described in Example 1. After inoculation the packed bed column is fed an appropriate growth substrate and nutrients to promote biofilm accumulation in the column. As biofilm develops, the corresponding pressure drop and flow rate across the reactor can be continuously monitored. Changes in media hydraulic conductivity, permeability and porosity are subsequently computed.

Results

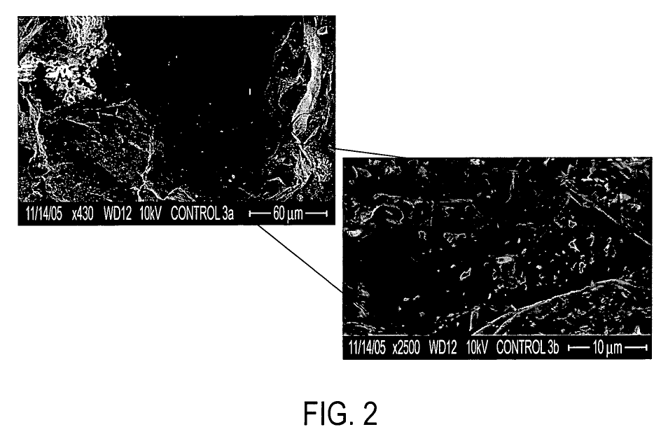

A Shewanella frigidamarina biofilm in a brea sand stone core was grown in the high pressure core pressure system under 1200 psi pressure and 35° C. conditions. FIG. 2 is a micrograph from a Scanning Electron Microscope showing the presence of significant biofilm growth in the core after a period of about 100 hours. A 2.5 order of magnitude drop in the core permeability was observed due to biofilm accumulation over 100 hours. As can be seen in the micrograph, the biofilm survived the challenge by supercritical CO₂. (Additional data not shown).

All publications, patents and patent applications discussed herein are incorporated by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method of using a microbial biofilm barrier to seal a gas leakage in a geological formation containing sequestered gas, comprising applying one or more microbial species to said geological formation and administering a growth substrate to said one or more microbial species to induce formation of a microbial biofilm which seals said gas leakage.

2. The method of claim 1, wherein said one or more microbial species are capable of growth at 40° to 60° C., about 5% salinity and about 100 atmospheres of pressure.

3. The method of claim 2, wherein said one or more microbial species are native to said geological formation.

4. The method of claim 1, wherein said one or more microbial species are capable of inducing calcium carbonate precipitation.

5. The method of claim 1, wherein said one or more microbial species are bacterial species which are starved prior to administration of said growth substrate.

6. The method of claim 5, wherein said one or more bacterial species are Shewanella sp.

7. The method of claim 1, wherein said geological formation is selected from the group consisting of a saline aquifer, a deep coal bed, a depleted oil reservoir, a depleted natural gas reservoir, and a salt cavern.

8. The method of claim 7, wherein said geological formation is a saline aquifer.

9. The method of claim 7, wherein said saline aquifer comprises one or more aquitard traps.

10. The method of claim 9, wherein said biofilm reduces leakage of sequestered gas through said one or more aquitard traps.

11. The method of claim 10, further wherein said biofilm provides a zone of reduced permeability in strata overlying said one or more aquitard traps to mitigate migration of gas leaks which have penetrated said one or more aquitard traps.

12. The method of claim 1, wherein said biofilm reduces leakage of sequestered gas in a region surrounding a point of injection of said sequestered gas.

13. The method of claim 1, wherein said sequestered gas is CO₂.

14. The method of claim 13, wherein said CO₂ is supercritical CO₂.

15. The method of claim 1, wherein said geological formation is man-made or natural.

16. The method of claim 1, wherein said biofilm partially seals said gas leakage.

17. The method of claim 1, wherein said biofilm completely seals said gas leakage.

18. A method of sequestering CO₂ in a geological formation, comprising:
a). injecting supercritical CO₂ in said geological formation;
b). adding one or more microbial species capable of forming a barrier at site of supercritical CO₂ injection; and
c). inducing formation of said barrier, wherein said barrier mitigates leakage of sequestered CO₂.

19. The method of claim 18, wherein the formation of said barrier is induced by starving said one or more microbial species followed by administration of a growth substrate.

20. The method of claim 18, wherein said one or more microbial species are capable of forming said barrier at 40° to 60° C., about 5% salinity and about 100 atmospheres of pressure.

21. The method of claim 18, wherein said one or more microbial species are native to said geological formation.

22. The method of claim 18, wherein said barrier comprises calcium carbonate precipitation.

23. The method of claim 18, wherein said one or more microbial species are bacterial species.

24. The method of claim 23, wherein said one or more bacterial species are Shewanella sp.

25. The method of claim 18, wherein said geological formation is selected from the group consisting of a saline aquifer, a deep coal bed, a depleted oil reservoir, a depleted natural gas reservoir, and a salt cavern.

26. The method of claim 25, wherein said geological formation is a saline aquifer.

27. The method of claim 26, wherein said saline aquifer comprises one or more aquitard traps.

28. The method of claim 27, wherein said barrier reduces leakage of the sequestered CO₂ through said one or more aquitard traps.

29. The method of claim 28, further wherein said barrier provides a zone of reduced permeability in strata overlying said one or more aquitard traps to mitigate migration of the sequestered CO₂, wherein the sequestered CO₂ has penetrated said one or more aquitard traps.

30. The method of claim 18, wherein said barrier reduces leakage of the sequestered CO₂ in a region surrounding said point of injection of the sequestered CO₂.

31. The method of claim 18, wherein said sequestered CO₂ is supercritical CO₂.

32. The method of claim 18, wherein said geological formation is man-made or natural.

33. The method of claim 18, wherein said barrier partially seals said gas leakage.

34. The method of claim 1, wherein said barrier completely seals said gas leakage.
